(19) United States
(12) Patent Application Publication
(10) Pub. No.: US 2010/0198209 A1
(43) Pub. Date: Aug. 5, 2010

(54) Hemorrhoid Therapy and Method

(76) Inventor: Joseph M. Tartaglia, Morgan Hill, CA (US)

Correspondence Address: SHAY GLENN LLP, 2755 CAMPUS DRIVE, SUITE 210, SAN MATEO, CA, 94403, US

(21) Appl. No.: 12/698,010

(22) Filed: Feb. 1, 2010

Related U.S. Application Data
(60) Provisional application No. 61/148,748, filed on Jan. 30, 2009.

(51) Int. Cl. A61B 18/04

(52) U.S. Cl. 606/27

(57) ABSTRACT

Methods and apparatus for treating hemorrhoids are provided. One feature of a vapor therapy apparatus is generating vapor with an electrode array in an elongate shaft. The apparatus may include a vapor delivery needle for delivering high temperature condensable vapor to a hemorrhoid. In some embodiments, the delivery needle is inserted into a hemorrhoid and a vapor of approximately 104-120 degrees Celsius is delivered into the hemorrhoid. The vapor can be delivered for approximately 1-5 seconds.

### CROSS-REFERENCE TO RELATED APPLICATIONS

This application claims the benefit under 35 U.S.C. 119 of U.S. Provisional Patent Application No. 61/148,748, filed Jan. 30, 2009, titled “Hemorrhoid Therapy and Method”. This application is herein incorporated by reference in its entirety.

### INCORPORATION BY REFERENCE

All publications, including patents and patent applications, mentioned in this specification are herein incorporated by reference in their entirety to the same extent as if each individual publication was specifically and individually indicated to be incorporated by reference.

### FIELD OF THE INVENTION

The invention relates generally to systems and their methods of use to treat venous insufficiency in the area of the anus and in the rectum. More particularly, the invention relates to vapor treatment of a hemorrhoid vein to reduce the inner diameter of the vein and create a luminal cellular response to reduce and/or eliminate the blood flow through the vein. The invention is generally used to divert the flow of blood from an insufficient vein to a vein that is sufficient.

### BACKGROUND OF THE INVENTION

Hemorrhoids, which are also called piles, are swollen and inflamed veins in the anus and rectum. They may result from straining during a bowel movement or the increased pressure on these veins during pregnancy, among other causes. Hemorrhoids are common. By age 50, about half of adults have had to deal with the itching, bleeding and pain that often signal the presence of hemorrhoids.

Hemorrhoid symptoms usually depend on the location. Two main categories are internal hemorrhoids and external hemorrhoids. Internal hemorrhoids cannot usually be seen or felt. Internal hemorrhoids may be caused by straining or irritation from passing stool, which can injure the vein's delicate surface and cause it to bleed. The patient may notice small amounts of bright red blood on toilet tissue or in the toilet bowl water. Because internal anal membranes lack pain-sensitive nerve fibers, these hemorrhoids usually don't cause discomfort. Occasionally, straining can push an internal hemorrhoid through the anal opening. If a hemorrhoid remains displaced (prolapsed), it can cause pain and irritation.

External hemorrhoids tend to be painful. Sometimes blood may pool in an external hemorrhoid and form a clot (thrombus), causing severe pain, swelling and inflammation. When irritated, external hemorrhoids can itch or bleed.

If hemorrhoids are producing only mild discomfort, the physician may suggest over-the-counter creams, ointments or pads containing witch hazel or a topical anti-inflammatory agent containing hydrocortisone. This local treatment, in combination with daily warm baths, may relieve the symptoms; but for many this is only a stop gap measure as the condition will worsen.

For persistent bleeding or painful hemorrhoids, other therapies are available. In rubber band therapy, the physician places one or two tiny rubber bands around the base of an internal hemorrhoid to cut off its circulation and the hemorrhoid falls off. In sclerotherapy, the physician injects a chemical solution around the blood vessel to shrink the hemorrhoid.

Some therapies and proposed therapies use some form of energy to modify the hemorrhoid vein. For example, one- or two-second bursts of infrared light have been used to cut off circulation to small, bleeding hemorrhoids. Also, the use of laser or RF energy to alter characteristics of a hemorrhoid vein is described, e.g., in U.S. Pat. No. 6,024,742; U.S. Pat. No. 6,135,997 and U.S. Pat. No. 6,139,527.

If other procedures haven't been successful or if the patient has large hemorrhoids, the physician can remove tissue in a procedure called hemorrhoidectomy. The surgery is done with either a local anesthetic combined with sedation, a spinal anesthetic, or a general anesthetic. Surgery can be performed on an outpatient basis or the patient may require an overnight hospital stay. Stapling is an alternative to hemorrhoidectomy. In this procedure, tissue is stapled to block blood flow to the hemorrhoidal tissue. While some experts point out that this procedure involves less pain than surgery and allows an earlier return to work, there are drawbacks as well. Stapling has been associated with a greater risk of hemorrhoidal recurrence and prolapse compared with open surgery.

There is a need for a safe, fast, effective and easy to administer therapeutic procedure to treat hemorrhoids. There is a need for a new therapy that is as effective as surgery, but which is minimally invasive and able to be performed in an office setting. This would allow the physician to administer therapy early in the progression of the condition, before the hemorrhoids become very painful (external hemorrhoids) or bleed profusely (internal and external hemorrhoids).

### SUMMARY OF THE INVENTION

In one embodiment, a method of treating a hemorrhoid comprises inserting a vapor therapy device into the hemorrhoid, and delivering a high temperature vapor from the vapor therapy device to the hemorrhoid. The vapor therapy device can include an elongate shaft, an electrode array, a pump, an RF generator, a fluid reservoir, and a delivery needle with vapor exit ports disposed therein. In some embodiments, fluid is pumped into the electrode array to generate a high temperature condensable vapor. In some embodiments, the electrode array is disposed within the elongate shaft.

The method can further comprise generating the high temperature vapor within a shaft of the vapor therapy device. In other embodiments, the method comprises generating the high temperature vapor with an electrode array disposed within the shaft of the vapor therapy device.

In one embodiment, the inserting step further comprises inserting a delivery needle of the vapor therapy device into the hemorrhoid. The hemorrhoid can be an internal or an external hemorrhoid, for example. In some embodiments, the delivering step further comprises delivering the high temperature vapor from the vapor therapy device to an interior of the hemorrhoid.

In some embodiments, the high temperature vapor is approximately 104-120 degrees Celsius.

In one embodiment, the method further comprises delivering the high temperature vapor to the hemorrhoid for approximately 1-2 seconds.

In another embodiment, the delivering step further comprises delivering the high temperature vapor from the vapor therapy device to an exterior of the hemorrhoid.

### BRIEF DESCRIPTION OF THE DRAWINGS

FIG. 1 illustrates a vapor therapy system configured to treat hemorrhoids.

### DETAILED DESCRIPTION OF THE INVENTION

Application of vapor to tissue can cause the temperature of the vein wall to be elevated for a certain amount of time, causing collagen contraction, an inflammatory response and endothelial damage. Collagen contraction occurs from an elevated temperature over a short time, below the temperature that causes tissue desiccation. The cellular damage that occurs during this process, as well as the denudation of the more sensitive endothelial cells, facilitates collapse of the vein and eventual closure as rapid organization within the vein lumen forms a fibrotic seal.

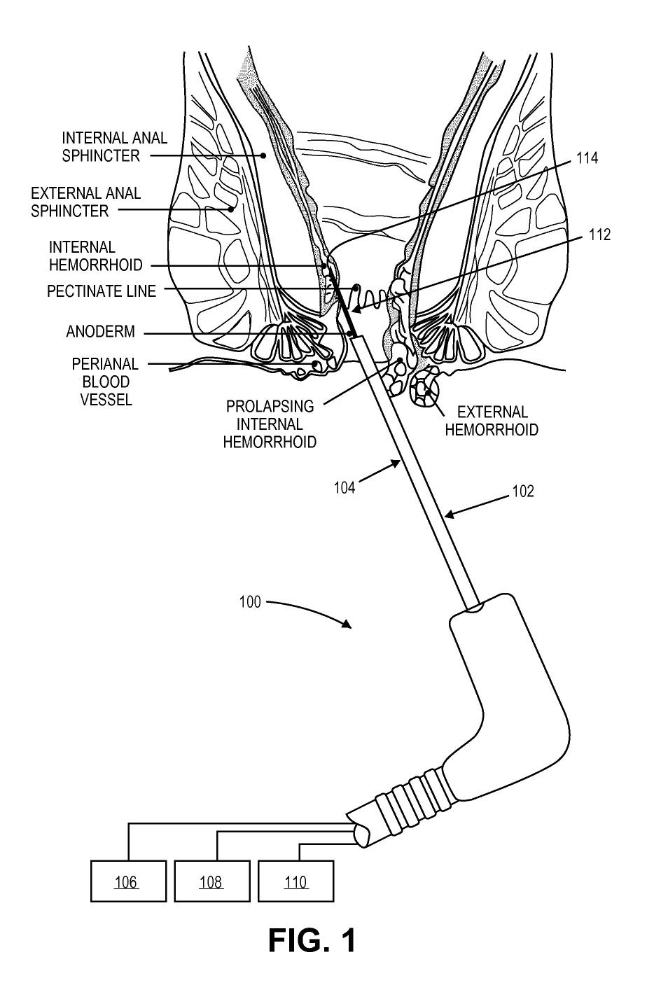

FIG. 1 illustrates a vapor therapy system 100, including shaft 102, electrode array 104, pump 106, RF generator 108, fluid reservoir 110, delivery needle 112, and vapor exit port(s) 114. The vapor therapy system 100 shown in FIG. 1, with the inclusion of a vapor delivery needle 112, is configured for accessing and treating small superficial veins and/or surface varicosities with a high temperature condensable vapor.

The electrode array 104 can be integrated into the shaft 102 of the vapor therapy system. In some embodiments, the electrode array can be integrated within a handle of the system, or alternatively, can be positioned outside of the vapor therapy system, such as in a remote boiler. The electrode array can be in fluid communication with the fluid reservoir 110 and with vapor exit port(s) 114 disposed within delivery needle 112, positioned near the distal end of the shaft 102. The electrode array can be a bipolar electrode or bipolar electrode array, a monopolar electrode or monopolar electrode array, or a combination of a bipolar and monopolar electrode array, for example. To generate a high temperature condensable vapor, fluid from fluid reservoir 110 can be delivered to the electrode array 104 with a pump 106. The electrode array can apply energy from RF generator 108 to the fluid to generate a high temperature condensable vapor. The vapor can then be delivered to tissue through exit port(s) 114 in the delivery needle 112. Further details on the electrode array and generation of high temperature condensable vapor can be found in U.S. Provisional Appln. No. 61/228,298, filed Jul. 24, 2009, titled, “Hot Tip Vein Therapy Device”. This application is incorporated herein in its entirety.

Shaft 102 can be a rigid or semi-rigid elongate body, and can house electrode array 104, as described above. The delivery needle 112 can be disposed on a distal end of the shaft and be in fluid communication with the electrode array and fluid reservoir 110. The shaft can be sized and configured to deliver vapor to both internal and external hemorrhoids. In some embodiments, the shaft is approximately 6″ in length.

By using superheated vapor, temperatures are essentially self-limiting to that just above boiling; the vapor exiting the distal tip is approximately 104-120° C. This permits heating of tissue entirely without desiccation or charring. Further, vapor injected into a hemorrhoid is contained by the vein wall, thus limiting convective heating of adjacent tissues. Lastly, the even distribution of vapor within the lumen minimizes the risk of ‘hot spots’ as an even temperature is provided to the entire surface simultaneously. Another significant benefit of vapor treatment is that it flows to the internal surfaces of the vein from the pressure created as it exits the distal tip, and therefore does not require external compression of the vein, as other endovenous treatments require. This enhances efficacy by reducing procedural inconsistency and complexity.

Methods of treating hemorrhoids are provided. In one embodiment, a vapor therapy system is placed in close proximity to a hemorrhoid. The hemorrhoid can be an internal hemorrhoid within the anus, or an external hemorrhoid. The vapor therapy system can be inserted into the hemorrhoid to treat the tissue. In some embodiments, a delivery needle of the vapor therapy system can pierce the distended or swollen vein of the hemorrhoid in one or more places. A high temperature steam or other vapor can then be delivered from the vapor therapy device to the interior of the hemorrhoid through the delivery needle, to heat shrink the vessel wall and denude the endothelial lining. In some embodiments, the high temperature vapor is generated by heating fluid within the shaft of the vapor therapy system with an electrode array before being delivered to the hemorrhoid. In another embodiment, the vapor is formed outside of the vapor therapy system in a remote boiler. In some embodiments, the temperature of the vapor can be between approximately 104-130° C. and preferably between 104-120° C. In some embodiments, the vapor is delivered to a hemorrhoid for approximately 1-2 seconds. In other embodiments, the vapor can be delivered for longer periods of time, such as 1-10 seconds, depending on the temperature of the vapor and the size of the hemorrhoid.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A method of treating a hemorrhoid, comprising:
inserting a vapor therapy device into the hemorrhoid; and
delivering a high temperature vapor from the vapor therapy device to the hemorrhoid.

2. The method of claim 1 further comprising generating the high temperature vapor within a shaft of the vapor therapy device.

3. The method of claim 2 further comprising generating the high temperature vapor with an electrode array disposed within the shaft of the vapor therapy device.

4. The method of claim 1, wherein the inserting step further comprises inserting a delivery needle of the vapor therapy device into the hemorrhoid.

5. The method of claim 1 wherein the high temperature vapor is approximately 104-120 degrees Celsius.

6. The method of claim 1 further comprising delivering the high temperature vapor to the hemorrhoid for approximately 1-2 seconds.

7. The method of claim 1 wherein delivering step further comprises delivering the high temperature vapor from the vapor therapy device to an interior of the hemorrhoid.

8. The method of claim 1 wherein delivering step further comprises delivering the high temperature vapor from the vapor therapy device to an exterior of the hemorrhoid.

9. The method of claim 1 wherein the hemorrhoid is an internal hemorrhoid.

10. The method of claim 1 wherein the hemorrhoid is an external hemorrhoid.
